(19) United States
(12) Patent Application Publication
(10) Pub. No.: US 2011/0123979 A1
(43) Pub. Date: May 26, 2011

(54) DETECTION OF MICROORGANISMS

(76) Inventors: Peter Salmon, Berkshire (GB); Robert Stewart Anthony, Berkshire (GB)

(21) Appl. No.: 12/981,345

(22) Filed: Dec. 29, 2010

Related U.S. Application Data
(63) Continuation-in-part of application No. PCT/GB2009/001698, filed on Jul. 9, 2009.

(30) Foreign Application Priority Data
Jul. 16, 2008 (GB) 0812999.1
Dec. 31, 2009 (GB) 0922725.7

(51) Int. Cl. C12Q 1/70; C12Q 1/04; C12Q 1/68

(52) U.S. Cl. 435/5; 435/34; 435/6.15

(57) ABSTRACT

A method of collecting, detecting and enumerating microorganisms in a fluid comprising subjecting a sample of the fluid to dielectrophoresis and collecting the microorganisms onto a microelectrode, scanning the microelectrode using a scanning laser and determining the number of microorganisms present on the microelectrode. Alternatively, the microorganisms may be spun onto a substrate which has been pre-treated with a polycationic electrolyte.

### CROSS-REFERENCE TO RELATED APPLICATION(S)

This application is a continuation-in-part of International Patent Application No. PCT/GB2009/001698, filed Jul. 9, 2009, which claims priority to and the benefit of British Application No. GB 0812999.1, filed Jul. 16, 2008, and also claims priority to British Application No. GB 0922725.7, filed Dec. 31, 2009, the entire contents of which are incorporated herein.

### SUMMARY

The present invention relates to a method and apparatus for collecting, detecting and enumerating microorganism in fluids. In particular, it relates to the rapid detection and enumeration of microorganisms in mammalian fluids such as blood or blood products.

A previously used method for detecting and enumerating microbial contamination of blood or blood products involved culturing a sample of blood or blood product. However, such a method was too slow, requiring several hours/days incubation. It was also too insensitive to be of any practical use. Elder, A F et al found that up to 50% of bacterially contaminated platelets may escape detection by culture at 24 hours (see Transfusion, (2007), 47, 1134).

A solid phase laser scanner has been used to enumerate bacteria in water. Broadway, S C et al in Appl Environ Microbial, (2003), 69(7), 4272-4273 described the rapid staining and enumeration of small numbers of bacteria in water using solid-phase laser cytometry. In order to determine the number of bacteria, a sample of water was filtered through a black polycarbonate membrane and then an overlay of SYBR Green I dye was applied to the filter. After incubation and removal of the stain, the membrane was dried and chilled prior to laser scanning. This method suffers from the disadvantage that interaction of the dye with the membrane filter produces non-specific spots of stain. In addition, microscopic examination of the membrane is usually necessary to identify possible non-specific stains. Other particulates in the water may take up the dye and become trapped on the membrane resulting in false positive counts.

Dielectrophoresis (DEP), which is the motion of electrically neutral particles or cells in response to a non-uniform electric field and can occur equally well in both DC and AC electric fields, has been used to quantify the number of particles in a liquid sample. Allsopp, D W E et al in J Phys D: App Phys, (1999), 32, 1066-1074 described an impedance technique for measuring dielectrophoretic collection of microbiological particles. The authors showed that measurement of the impedance change resulting from the collection of microbiological particles at coplanar electrodes enabled them to quantify the concentration of particles collected under positive dielectrophoretic force. The disadvantages of this method are a) low sensitivity in that at least 10⁵ ml bacteria are required to incur a measurable impedance change, b) inflexible sample conditions in that the bacteria must be suspended in a buffer with an extremely low conductivity. Furthermore the change in impedance does not correlate with an accurate bacterial count. The size and cell wall characteristics influence the magnitude of the impedance change.

In order to safe-guard the biological safety of blood or blood products a rapid screening technique is needed that will detect less than 1,000 bacteria per ml of sample, preferably less than 100 bacteria per ml. Unfortunately, measuring impedance change is not sensitive enough and cannot detect such low levels of contamination.

According to an aspect of the invention, there is provided a method of collecting and detecting microorganisms in a fluid, comprising the steps of treating a substrate with a polycationic electrolyte, causing microorganisms to be adhered to the treated substrate and scanning the substrate with a scanning laser in order to count and/or detect the microorganisms.

The substrate may be polycationic treated (e.g. polycationic coated) glass. It may be other material.

Some (non-limiting) aspects of the invention are set out in the appendant claims.

According to a further aspect of the invention, there is provided a method of collecting and detecting microorganisms in a fluid comprising the steps of subjecting a sample of said fluid to dielectrophoresis and collecting the microorganisms onto a microelectrode, scanning the microelectrode using a scanning laser and determining the number of microorganisms present on the microelectrode.

Using dielectrophoresis (DEP) and/or treating a substrate with a polycationic electrolyte can enable microorganisms to be attracted or deposited from a suspending fluid or sample into the focal plane of a scanning laser and photomultiplier tubes to rapidly detect and quantify microbial contamination of the fluid.

DEP may optionally be used in conjunction with a polycationic means. The polycationic electrolyte may be a polycationic treated glass. DEP would then generally not be used as it would be unnecessary.

According to a further aspect of the invention, there is provided the use of dielectrophoresis and/or polycationic electrolytes in combination with laser scanning cytometry to collect and detect microorganisms in a fluid.

The method may be used for detecting microorganisms such as bacteria, viruses, yeasts, algae, protozoa and fungi.

The fluid may be any mammalian fluid such as urine or cerebrospinal fluid, however, the method is particularly useful for detecting microorganisms in blood or blood products, such as platelets.

### BRIEF DESCRIPTION OF THE DRAWINGS

Embodiments of the invention will now be described, by way of example only, with reference to the accompanying schematic drawings, in which:

FIG. 1 shows a microslide arrangement with six cells;

FIG. 2 shows the concept of DEP;

FIG. 3 shows an electrode placed in a laser scanner; and

FIG. 4 shows an electrode in a well.

### DETAILED DESCRIPTION

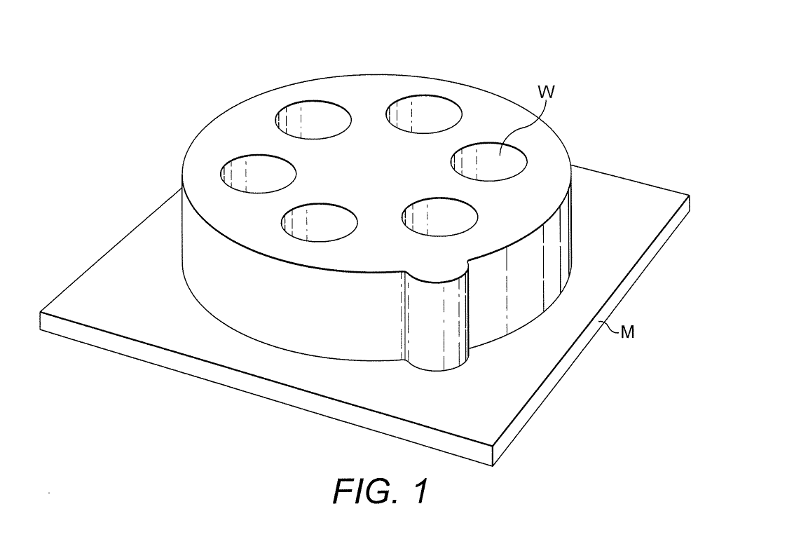

Referring to FIG. 1, bacteria (or other organisms), separated from platelets by specific lysis and solubilisation of platelets, are stained with a fluorophore and spun onto the glass base of a well W in a microslide arrangement M with six wells (FIG. 1). The glass surface is pre-treated with a polycationic electrolyte or polyelectrolyte to facilitate bacterial adhesion and retention during analysis in a laser scanning cytometer. During analysis the microslide is inverted and bacteria adherent on the underside of the glass are scanned from above by a scanning laser and light detection system. Fixing the bacteria to the glass retains them within the precise focal plane of the scanning optics. The light pattern emitted by the stained sample can then be discriminated on various parameters by appropriate software, as will be evident to those skilled in the art, to identify and enumerate the bacteria in the sample.

Taking some aspects in more detail, firstly, to a volume of platelets (nominally 1 mL) is added an equal volume of a lysis solution (8M urea, 2% w/v Triton X-100, 2% w/v polyethylene glycol 6000, 320 mM manitol). Mixing achieves complete lysis and solubilisation of platelet structural proteins.

The platelet lysate is then centrifuged for a sufficient time in a centrifuge tube (Salivette tube, Sarstedt) with a narrow base to permit efficient bacterial collection.

The lysate is poured off and the bacteria stained with a fluorescent dye. Staining may be by the following categories of fluorescent dye/substrate
a. nuclear stain
b. protein stain
c. fluorophore labelled antibiotics, eg polymixin B for gram -ye bacteria and vancomycin for gram+ve bacteria. or
d. whole cell stains including lipophilic stains.

The bacteria may be washed in an isosmotic buffer by centrifugation to remove unbound fluorescent stain in the supernatant

The pelleted bacteria are then transferred to a well W on the glass microslide M. The glass on the microslide is coated during manufacture with a cationic polyelectrolyte examples of which are poly-L-lysine, polyethyleneimine.

The microslide is placed in a dedicated centrifuge bucket insert and spun in a swing bucket rotor to ensure even distribution of the bacteria over the surface of the glass. Of course, any convenient spinning method may be used. It is spun for a sufficient time to ensure as even as possible distribution. Trial and error can easily be used for this.

The microslide is inverted and placed in a laser scanning cytometer.

Fluorescent pattern data from the scan is then discriminated and analysed by dedicated software to produce an accurate count or bacteria in the sample.

In order to separate any contaminating microorganisms, prior to subjecting the sample of fluid to dielectrophoresis or other treatment, a lysis solution is preferably added in order to lyse any mammalian cells present. The contaminating microorganism may then be separated using centrifugation, after which they are stained or labelled so that they will fluoresce when subjected to the scanning laser. Suitable stains or labels may include non-specific nuclear dyes such as SYBR Green I and acridine orange, metabolic substrates that become fluorescent through enzymatic activity, antibodies, including monoclonal antibodies, to microbial proteins, or molecular probes which can hybridise to microbial genetic material or a combination of these.

In a further embodiment, the labelled contaminating microorganisms suspended in a fluid are then loaded onto a microelectrode. The microelectrode comprises at least one pair of adjacent co-planar electrodes of micron dimensions for electrode width or gap size, and is supported on a substrate. The microelectrodes can be manufactured in various metals, including gold and aluminium. The substrate material is transparent and preferably of low autofluorescence, for example glass or plastic. The microelectrode may be manufactured by micro-fabrication technology employing photolithography or by printing metal ink technology or by printing and electrode plating technology or by a combination of these methods.

The substrate material may be transparent and preferably of low autofluorescence, for example glass or plastic. Where glass is used, the substrate material may be pre-treated with a cationic polyelectrolyte (i.e. polycationic) to ensure that bacteria collected by DEP remain securely attached onto the substrate following removal of the DEP generating current. However, DEP generally will not be used where bacteria (or other microorganisms) are spun onto polycationic treated glass or other substrate.

Examples of suitable cationic polyelectrolytes may include poly-L-lysine or polyethyleneimine or other polycation electrolytes as appropriate. Pre-treatment involves immersion of the substrate material in a solution of the cationic electrolyte followed by surface drying in a stream of sterile air.

In further example of a low skill rapid preparation using silica micro-beads to stabilise the bacterial pellet and reduce the loss in counts through handling, lysis solution containing silica micro-beads (0.5 μn diameter) with a chemically modified anionic surface was added to platelets (1 mL) in a 2 mL micro-centrifuge tube and centrifuged at 7500× g for 2 min. The supernatant was poured off, the contaminating bacteria are effectively retained at the bottom of the micro-centrifuge tube by the silica bead pellet. The lysis procedure and centrifugation step were repeated on the bacterial pellet and the bacteria re-suspended. An aliquot of bacterial suspension was transferred to a well in the microslide and stained with a fluorescent double stranded DNA dye. The microslide was centrifuged at 2,500× g for 5 min and scanned directly by a laser-scanner apparatus of a suitable type, such as ‘Bac-Detect’.

The microelectrode may be placed in the bottom of a well, which may form part of a 96-well plate. In the well, the fluid remains static during DEP collection of the microorganism. Alternatively, the microelectrode may be placed in a flow through chamber where the suspending fluid containing the microorganisms is passed over the microelectrode on one side of the chamber in order to facilitate DEP collection from a larger sample. The microelectrode structure in these examples would normally be of a co-planar type. Alternatively, for a flowing sample, the microelectrode may be of a grid construction where a series of insulated grids are aligned to allow the passage of fluid.

Dielectrophoretic forces are produced across the microelectrode by an alternating current of fixed amplitude and wavelength. This may vary depending upon the conductivity of the suspending medium and the type of microorganisms to be collected. The signal may be a sine wave or a square wave and may or may not have a direct current offset depending upon the conductivity of the sample and the type of microorganism to be collected. DEP collects and concentrates any microorganisms suspended in the fluid onto the edge of the microelectrode.

Once the microorganisms have become trapped on the microelectrode, the current is switched off and the microelectrode is placed in the laser scanner. Scanning lasers and photomultiplier tube (PMT) detectors scan the area surrounding the microelectrode to excite the fluorescently labelled microorganisms and detect the emitted light. The wavelength used for laser scanning may vary depending on the fluorochrome used in the marker dye. In systems where two or more marker dyes of different excitation wavelengths are used, more than one source of laser light will be used.

The fluorescence intensity is collected at regular intervals by the PMTs and thresholding algorithms identify all the fluorescence intensities above background levels. Object intensity profiles enable the calculation of a range of morphological and fluorescent parameters to identify microorganisms collected onto the electrodes from the fluid sample.

The advantages of using dielectrophoresis and scanning laser cytometry to detect microbial contamination of fluids, such as blood or blood products, are that the preparation technique is simple, several samples can be analysed simultaneously and a true real-time analysis of contamination can be obtained rapidly. Furthermore, no complex reagents are required and minimal waste is produced by virtue of using microelectrodes. Thus, a sensitive, high through-put real-time point of issue test for contamination is produced.

A further embodiment of the present invention will be further described by way of reference to the following example:

In one example, lysis solution (8M urea, 2% w/v Triton X-100, 2% w/v polyethylene glycol 6000, 320 mM manitol) was added to human blood platelets (1 ml) in a Salivette tube (Sarstedt). The tube was centrifuged at 4,500× g for 15 min and the supernatant poured off. Contaminating bacteria, trapped in the restricted bottom of the Salivette tube were stained by the addition of Sybr green stain such that the final concentration of the stain was 1:2000. The sample was stained for 5 min and washed with a volume of isosmotic solution with centrifugation. The collected contaminating bacteria were transferred in a volume (50 μl) to a well in the microslide. The microslide is centrifuged at 2,500× g for 5 min, inverted and placed on the scanning drawer of a Bac-Detect laser scanner (Bac-Detect is available from Blood Analysis Ltd, PO BOX 71, Slough SL2 3SE). Laser scanning of the collected bacterial was initiated. The results were displayed by the software as pass or fail depending on the level of bacterial contamination detected in the platelet sample. Alternatively they can be displayed as an exact bacterial count and the bacteria visualised by an image of the scanning surface.

In another example, lysis solution (0.2 ml of 5% Triton, phosphate buffered saline (PBS) containing 10⁹ polyethylene imide (PEI) coated paramagnetic beads (10 mm diameter)) was added to human blood platelets (1 ml) in a microfuge tube (2 ml). The tube was centrifuged using Eppendorf centrifuge 5424 at 20,000× g for 2 min and then placed in a magnetic particle separator (mps). The beads and contaminating bacteria were allowed to collect on the wall of the tube and the lysate supernatant was poured off. The tube was removed from the mps and staining solution (2 μl of Sybr green 1:1,000 in 50 μl quarter strength Ringers solution) was added. The tube was incubated at room temperature for 5 minutes in the dark. The stained sample was the pipetted into a well in a dielectrophoretic microelectrode chip and connected to an alternating current (AC) signal of 100 KHz, 10 V amplitude for 10 minutes. The AC signal source was disconnected, the microelectrode chip was inverted in the scanning holder and loaded into a Bac-Detect laser scanner (Bac-Detect is available from Blood Analysis Ltd, PO BOX 71, Slough SL2 3SE). Laser scanning of the DEP collected bacterial was initiated. The results were displayed by the software as pass or fail depending on the level of bacterial contamination detected in the platelet sample. Alternatively they can be displayed as an exact bacterial count and the bacteria visualised by an image of the scanning surface.

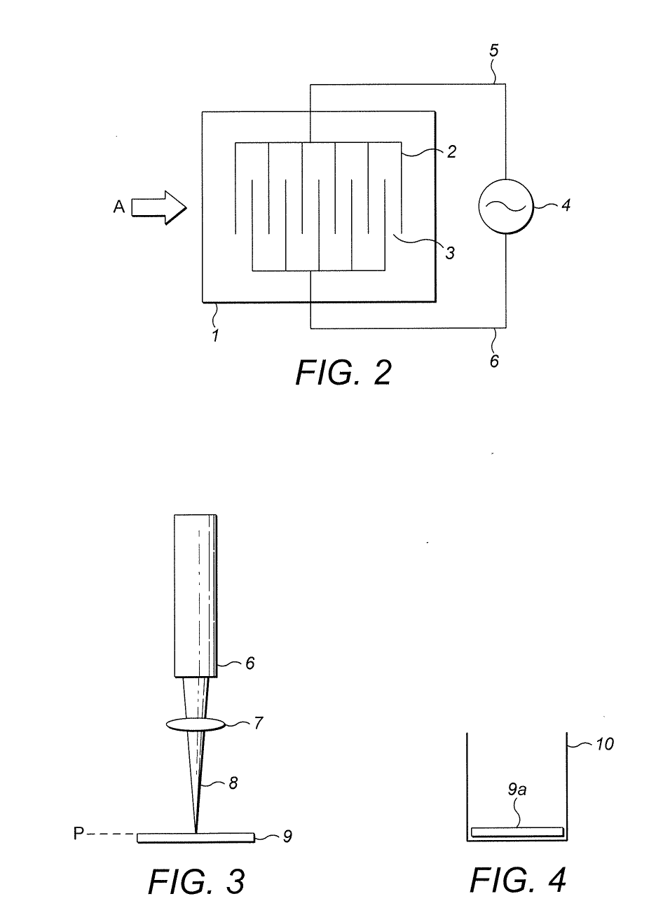

FIG. 2 shows DEP schematically. A sample A is passed across a substrate 1 having printed upon it, or otherwise formed upon it, a microelectrode structure comprising interdigitated electrodes 2 and 3. AC current 4 is generated and applied to the electrode via connections 5 and 6 to the respective electrodes 2 and 3. The electrodes are of micron dimensions and are energised with the voltage of a predetermined frequency using AC generator 4. The relevant particles (such as bacteria, biological cells and so on) collect on the electrode array and then, after the deposition process, the substrate can be analysed by visual inspection using microscopes or otherwise to count the number of particles and therefore information about the type and/or concentration of particles can be determined.

FIG. 3 shows, again very schematically, a scanning laser 6 and focusing optics 7 focussing a laser beam 8 onto a microelectrode 9. The optics may be an integral part of the laser, or separate. Scanning means, for causing the beam to scan relative to the sample may be included. The arrangement is preferably such that the sample is at the precise focal plane P of the scanning optics.

DEP may be used to collect microorganisms onto the edge of the electrode as discussed and the laser then scans to detect these, or in the embodiment of FIG. 1 or some other embodiments the microorganisms are collected onto polycationic electrolytes.

FIG. 4 shows a microelectrode 9a within a well 10.

What is claimed is:

1. A method of collecting and detecting microorganisms in a fluid, comprising the steps of treating a substrate with a polycationic electrolyte, causing microorganisms to be adhered to the treated substrate and scanning the substrate with a scanning laser in order to count and/or detect the microorganisms.

2. A method as claimed in claim 1, wherein the microorganisms are fixed to the substrate at the focal plane of the scanning laser.

3. A method as claimed in claim 1, wherein the microorganisms are spun onto the substrate.

4. A method as claimed in claim 1, wherein the microorganisms are stained with a fluorophore.

5. A method as claimed in claim 1, wherein the fluid contains platelets and the microorganisms are separated from platelets by specific lysis and solubilisation of platelets.

6. A method as claimed in claim 1, wherein the microorganisms are bacteria.

7. A method as claimed in claim 1, wherein the substrate is a slide provided with one or more wells.

8. A method of using polycationic electrolytes to pre-treat a substrate used for collecting microorganisms.

9. A method of collecting and detecting microorganisms in a fluid comprising the following steps:
a) subjecting a sample of said fluid to dielectrophoresis and collecting the microorganisms onto a microelectrode; and
b) scanning the microelectrode using a scanning laser and determining the number of microorganisms present on the microelectrode.

10. The method according to claim 9, wherein the microorganisms are selected from bacteria, viruses, yeasts, algae, protozoa, fungi and combinations thereof.

11. The method according to claim 9, wherein the sample of fluid is a mammalian fluid selected from urine, cerebrospinal fluid, blood and blood products.

12. The method according to claim 9 wherein, prior to subjecting the fluid sample to dielectrophoresis, a lysis solution is optionally added to the sample of fluid and any microorganisms present are separated by centrifugation.

13. The method according to claim 9, wherein the microorganisms present in the sample are stained or labelled.

14. The method according to claim 13, wherein the stain or label is selected from non-specific nuclear dyes, metabolic substrates that become fluorescent through enzymatic activity, antibodies to microbial proteins and molecular probes that hybridise to microbial genetic material.

15. The method according to claim 9, wherein in step b) the microelectrode and area surrounding the microelectrode are scanned using the scanning laser connected to a photomultiplier tube detector to excite fluorescently labelled microorganisms and detect emitted light.

16. The method according to claim 15, wherein means are provided for enumerating and/or identifying microorganisms collected onto the microelectrode from the fluid sample.

17. The method as claimed in claim 1, wherein the substrate is pre-treated with a cationic electrolyte.

18. A method of using dielectrophoresis in combination with laser scanning cytometry to collect, detect and optionally enumerate microorganisms in a fluid.

19. The method of claim 18, wherein the microorganisms are selected from bacteria, viruses, yeasts, algae, protozoa, fungi and combinations thereof.

20. The method of claim 18, wherein the fluid is a mammalian fluid selected from urine, cerebrospinal fluid, blood and blood products.
